(19) United States
(12) Patent Application Publication
(10) Pub. No.: US 2013/0321579 A1
(43) Pub. Date: Dec. 5, 2013

(54) System and Method for Scanning and Analyzing a Users Ergonomic Characteristics

(71) Applicant: Darcy Paul Firkus, Franktown, CO (US)

(72) Inventor: Darcy Paul Firkus, Franktown, CO (US)

(21) Appl. No.: 13/487,628

(22) Filed: Jun. 4, 2012

(51) Int. Cl. H04N 13/02; H04N 5/33; H04N 7/18

(52) U.S. Cl. 348/46; 348/E13.074; 348/E07.085

(57) ABSTRACT

A system including a 3-D scanner adapted for connection to a user's workspace computer, including an infrared light source, an infrared light detector, a three-dimensional camera and an RGB camera. The 3-D scanner is operatively connected to a user's computer, and captures a three-dimensional image of a user's posture. An algorithm is also provided which is adapted to receive the three-dimensional image of the user's posture and to receive other information relating to the user's ergonomic characteristics. The algorithm is configured to analyze the user's posture and the other information relating to the user's ergonomic characteristics and to provide a report on the user's ergonomic attributes. The method of using the system is also disclosed.

### BRIEF DESCRIPTION OF THE DRAWINGS

FIG. 1 is a front view of a user's workstation, with a 3-D scanner installed at the workstation.

FIG. 2 is a side view of a user's workstation, with a 3-D scanner installed at the workstation.

FIG. 3 is a block diagram, showing the interrelation of the components of the 3-D scanning system.

FIG. 4 is a flowchart setting forth the steps of the method for scanning and analyzing a user's ergonomic characteristics.

### DETAILED DESCRIPTION OF THE DRAWINGS

Embodiments of a system and method for scanning and analyzing a user's ergonomic characteristics are shown and described. Generally, system comprises a 3-D scanner adapted for connection to a user's workspace computer, the 3-D scanner including an infrared light source, an infrared light detector, a three-dimensional camera, and at least one RGB camera. The 3-D scanner is operatively connected to a user's computer, and the 3-D scanner captures a three-dimensional image of a user's posture and/or the location of equipment situated on a user's workstation. Also provided is an algorithm configured to operate on the user's computer, the algorithm being adapted to receive the three-dimensional image of the user's posture and/or the location of equipment situated on a user's workstation and to receive other information relating to the user's ergonomic characteristics, said algorithm being further configured to analyze the user's posture and the other information relating to the user's ergonomic characteristics and to provide a report on the user's ergonomic attributes. Generally, the method comprises providing a 3-D scanner, the 3-D scanner comprising: an infrared light source, an infrared light detector, a three-dimensional camera, and at least one RGB camera; connecting the 3-D scanner to a user's computer; capturing by the 3-D scanner a three-dimensional image of a user's posture and/or the location of equipment situated on a user's workstation; providing an algorithm configured to operate on the user's computer, said algorithm adapted to receive the three-dimensional image of the user's posture and/or the location of equipment situated on a user's workstation and to receive other information relating to the user's ergonomic characteristics; analyzing by the algorithm the user's posture and/or the location of equipment situated on a user's workstation and the other information relating to the user's ergonomic characteristics and providing a report on the user's ergonomic attributes.

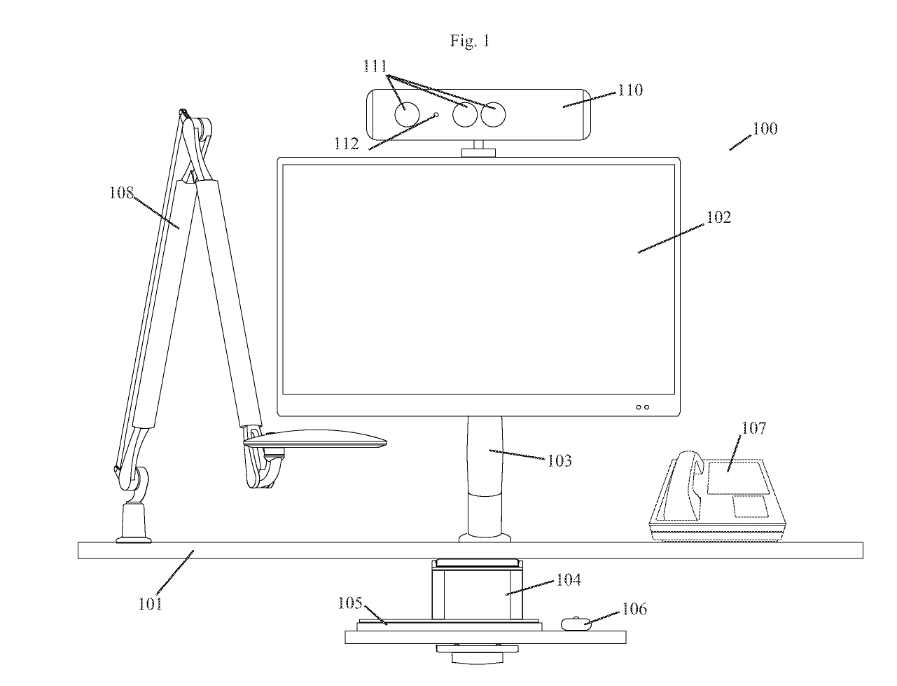

FIG. 1 shows a front view of a user's workstation, with a 3-D scanner installed at the workstation. The workstation 100, generally includes a desk surface 101, a computer monitor 102, supported by a monitor arm 103. A keyboard support 104, a keyboard 105 and mouse 106 may also be provided. A CPU (not shown) for the user's computer may also be provided. Other typical office equipment such as a telephone 107 and a lamp 108 may also be provided. One of ordinary skill in the art will readily appreciate that other devices or equipment commonly used in an office environment may also be provided at the user's workstation, including but not limited to calculators, scanners, file containers and the like.

Operatively connected to the CPU (not shown) of the user's computer is a 3-D scanner 110. 3-D scanner 110 has a plurality of apertures 111, which accommodate various optical or scanning components of the 3-D scanner, operation and functions of which will be discussed in greater detail infra. Optionally, 3-D scanner 110 can be provided with an indicator light 112, indicating to a user that the 3-D scanner is in operation. In FIG. 1, the 3-D scanner 110 is shown situated on top of the user's computer monitor 102. It should be appreciated that the 3-D scanner 110 can be positioned anywhere relative to the user's workstation to capture the required three-dimensional data for analysis. By way of example and without limitation, 3-D scanner 110 may be positioned above the user's workstation, looking down on it; it may be positioned to the side of the workstation to capture the 3-D image of the user and workstation in profile; it may be positioned in a plane parallel with the back of the user to capture the 3-D image of the workstation in a front view. The 3-D scanner may be positioned in any direction or orientation required to acquire 3-D images necessary for analysis.

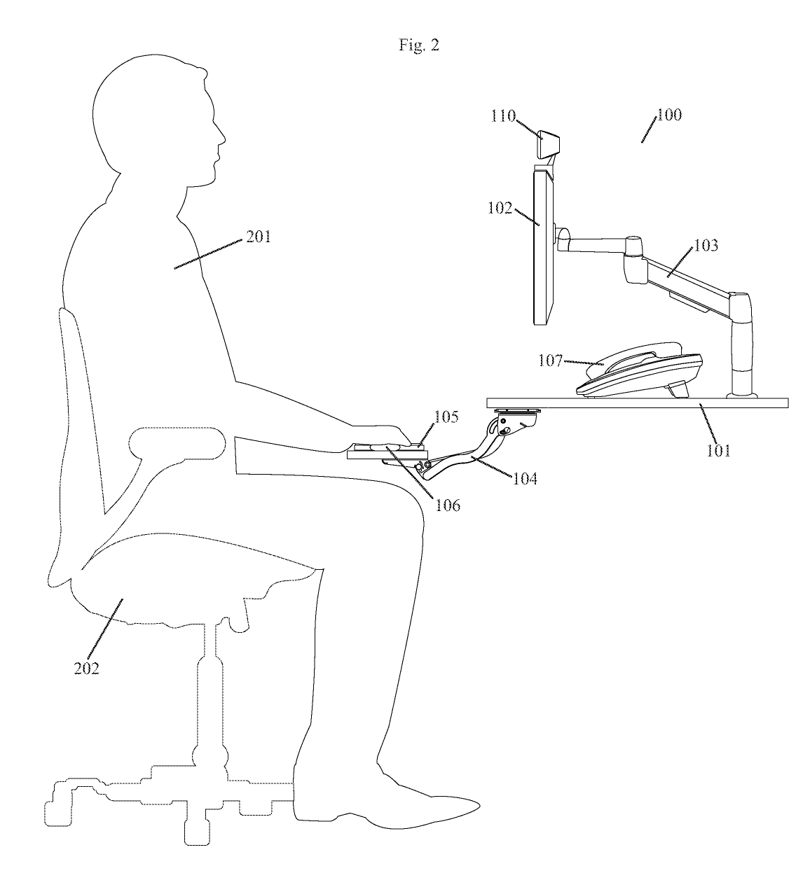

FIG. 2 shows a side view of a users workstation, with a 3-D scanner installed at the workstation. Like numerals are used to identify structure common to FIGS. 1 and 2. The workstation 100 is provided with desk surface 101, and computer monitor 102 mounted on arm 103. Keyboard support 104 supports keyboard 105 and mouse 106. Telephone 107 is disposed on desk surface 101 and 3-D scanner 110 is situated on top of monitor 102. Also shown in FIG. 2 are user 201 seated in chair 202. The 3-D scanner 110 captures three-dimensional images and data regarding user's 201 posture. The 3-D scanner 110 also captures three-dimensional mages and data regarding the positioning of equipment, such as for example, telephone 107 on the user's workstation and the relative positioning of the user to the equipment. As stated supra, the 3-D scanner 110 may be positioned anywhere relative to the user's workstation to capture the required three-dimensional data for analysis. By way of example and without limitation, 3-D scanned 110 may be positioned above the user's workstation, looking down on it; it may be positioned to the side of the workstation to capture the 3-D image of the user and workstation in profile; it may be positioned in a plane parallel with the back of the user to capture the 3-D image of the workstation in a front view. The 3-D scanner may be positioned in any direction or orientation required to acquire 3-D images necessary for analysis.

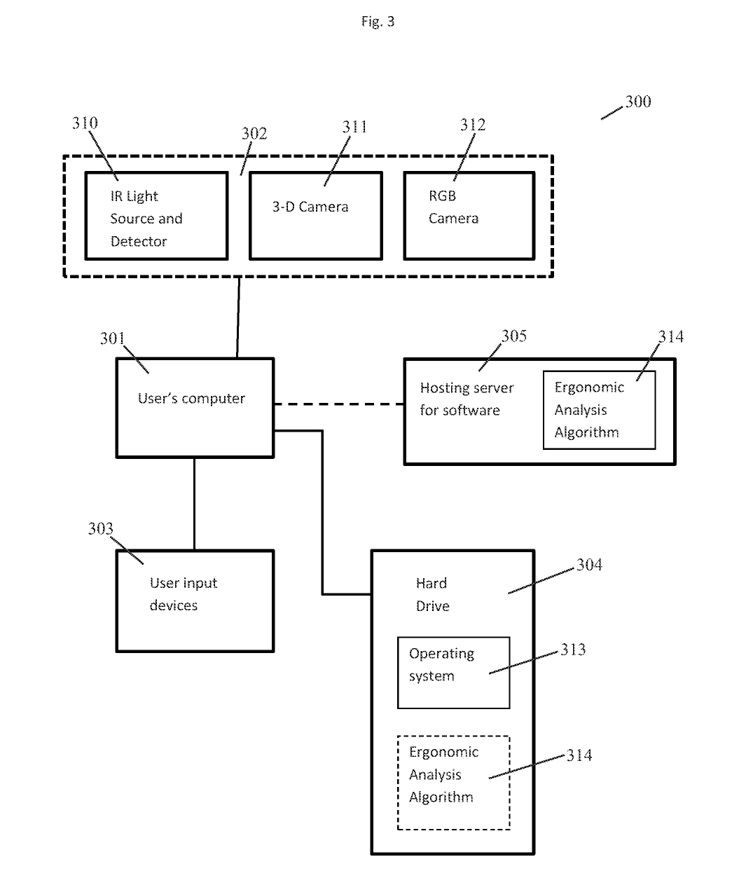

FIG. 3 shows a block diagram, showing the interrelation of the components of the 3-D scanning system and related analysis algorithm. The system 300 comprises a user's computer 301, the 3-D scanner 302, and at least one user input device 303. Also provided are hard drive 304, with associated data provided thereon, and optionally, network server 305 for hosting the ergonomic analysis algorithm. Each of these components will now be described in greater detail.

User's computer 301 may be the computer which user utilizes for everyday work at the workstation. Alternatively, user's computer 301 may be specially provided at the workstation for the analysis of the user's ergonomic characteristics. It should be appreciated that any type of computer may be used as the user's computer 301, so long as it is compatible with the software which implements the ergonomic analysis algorithm. By way of example and without limitation, user's computer 301 may be a PC, Mac or other computer operating a standard operating system. Furthermore, user's computer 301 may be a desktop computer, laptop computer, netbook, tablet, or any other form of computer. One of ordinary skill in the art will appreciate that all of the components typically included as part of a computer package are to be included within the user's computer 301. Thus, the user's computer 301 may be provided with a monitor for displaying information, a processor, memory, other peripherals, and user input devices which will be discussed in greater detail infra. All of these components are understood to be included within the user's computer 301. The user's computer 301, must be provided with a Universal Serial Bus (USB) or other type of connection capable of establishing a connection with the 3-D scanner 302. The form of this connection will depend on the nature of the connections on the 3-D scanner 302.

The 3-D scanner 302 is operatively connected to the user's computer 301 and is comprised of an infrared light source and detector 310, a 3-D camera 311 and an RGB camera 312. The structure and function of these components will now be discussed in detail. The infrared light source component of infrared light source and detector 310 emits infrared light onto the scene to be scanned. The sensor component of infrared light source and detector 310 detects the backscattered infrared light from the surface of one or more targets or objects in the scene and uses this detected infrared light to create a three-dimensional image of the scene. It should be appreciated that any infrared light source and detector known to the art and suitable for the application may be used in the 3-D scanner 302. Similarly, it should be appreciated that any 3-D camera or RGB camera known to the art and suitable for the application may be used as the 3-D camera 311 or the RGB camera 312.

Pulsed infrared light may be used to determine the distance of targets or objects in the scene by comparing the time between the outgoing infrared light pulse and corresponding incoming light pulse. Alternatively, the phase of the outgoing light pulse can be compared to the phase of the incoming light pulse and the determined phase shift can be used to determine the distance between the 3-D scanner 302 and targets or objects in the scene. In still another alternative embodiment, the intensity of the incoming infrared light pulse can be compared to the intensity of the outgoing infrared light pulse to determine the distance to targets or objects in the scene. In all of the aforementioned embodiments, the incoming infrared light may be captured by the detector component in the infrared light source and detector 310, by the 3-D camera 311 or by the RGB camera 312. One or more of these components may simultaneously capture the incoming infrared light, and thereby create multiple and distinct calculations of the three-dimensional image of the scene. Determining the contours of a three-dimensional scene in the manners set forth above is called time-of-flight analysis.

Other methods of determining the three-dimensional contours of a scene may also be used by the 3-D scanner. For example, the infrared light source component in the infrared light source and detector 310 may project a standard light pattern onto the scene, e.g. in the form of a grid or stripe pattern. When this pattern of light strikes the surface of targets or objects in the scene it becomes deformed, and the deformation of the pattern may be detected by the 3-D camera 311 or the RGB camera 312. This deformation can then be analyzed to determine distance between targets or objects in the scene and the 3-D scanner 302. Determining the contours of a three-dimensional scene in the manner set forth above is called structured light analysis.

In yet another method for determining the three-dimensional contours of a scene, the 3-D scanner 302 may be provided with two or more separate cameras. These cameras may be the 3-D camera 311 and the RGB camera 312. Also, more than one 3-D camera 311 or more than one RGB camera 312 may be provided. The two or more cameras provided in the 3-D scanner 302 view the scene from different angles, and thereby obtain visual stereo data. This distance between targets or objects in the scene and the 3-D scanner 302 can be determined from this visual stereo data. Determining the contours of a three-dimensional scene in the manner set forth above is called stereo image analysis.

It should be appreciated that a single one or multiple types of analysis may be used serially or simultaneously to determine the contours of a three-dimensional scene. The 3-D scanner 302 may be connected to the user's computer 301 by any conventional means known in the art. By way of example and without limitation, 3-D scanner 302 can be connected to the user's computer 301 by a Universal Serial Bus (USB) connection. Alternatively, the 3-D scanner 302 may be connected to the user's computer through a wired or wireless network connection. Any means of connection that facilitates the transfer of data from the 3-D scanner to the user's computer may be used.

Also connected to the user's computer 301 are user input devices 303. The user input devices 303 will usually take the form of a keyboard and mouse. However, it should be appreciated that any device capable of taking input from the user and making it available for use by the computer is within the scope of the user input device 303. By way of example and without limitation, the user input device 303 may also be trackballs, microphones or any other device which allows a user to input information. The user input devices 303 allow a user to input information about their ergonomic characteristics, which information is then used by the ergonomic analysis algorithm in combination with the posture or equipment position data obtained from the 3-D scanner, in a manner discussed in greater detail infra.

The user's computer 301 may be provided with a hard drive 304. The hard drive 304 provides long-term storage of programs and data for use by the user's computer. It should be appreciated that any non-volatile memory system capable of storing data for access by the user's computer may be used as the hard drive, and such other systems fall within the scope of that term. Resident on the hard drive 304 is the operating system 313 for the user's computer 301. As stated supra, any of the commonly available operating systems that will support the 3-D scanner can be used as operating system 313. By way of example and without limitation, operating system 313 may be Windows operating system, Mac operating system, Linux or any other commonly available operating system.

Optionally provided on hard drive 304 is ergonomic analysis algorithm 314. In an alternate embodiment also shown in FIG. 3, a remote hosting server for software 305 may be provided with ergonomic analysis algorithm 314 resident thereon. Thus, the ergonomic analysis algorithm 314 may either be provided locally on the user's computer 301 or it may be provided remotely and accessed by the user's computer. Additionally, one of ordinary skill in the art will appreciate that a portion of the ergonomic analysis algorithm 314 could be resident on the user's computer and an additional portion could be accessed remotely on the hosting server 305. It should be appreciated that either a locally provided or remotely accessed copy of the ergonomic analysis algorithm is contemplated by this disclosure. The ergonomic analysis algorithm 314 receives from the 3-D scanner 302 three-dimensional data regarding the user's posture and/or three-dimensional data regarding the placement of equipment on the user's workstation. The ergonomic analysis algorithm 314 also receives information input by the user regarding the user's ergonomic characteristics. The ergonomic analysis algorithm analyzes this data and outputs a report on the user's ergonomic attributes. This method by which this is accomplished is described infra.

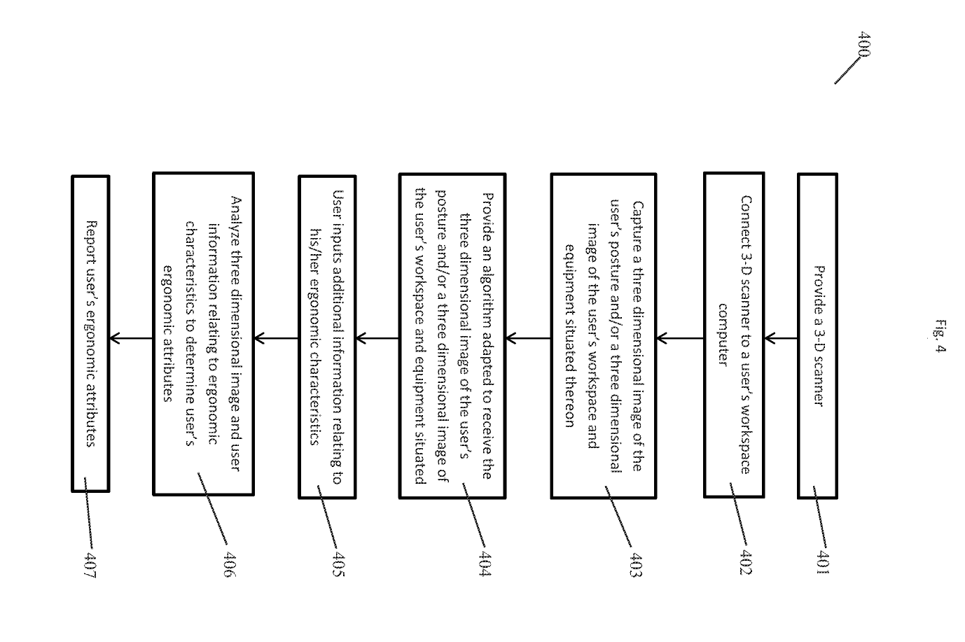

FIG. 4 shows a flowchart setting forth the steps of the method 400 for scanning and analyzing a user's ergonomic characteristics. In step 401, a 3-D scanner is provided. The 3-D scanner is constructed as set forth supra and includes an infrared light source; an infrared light detector; a three-dimensional camera; at least one RGB camera. In step 402, the 3-D scanner is operatively connected to the user's computer. This connection is made by any means known in the art for connecting computer peripherals to a computer.

In step 403, the 3-D scanner captures a three-dimensional image of a user's posture. In addition to, or in place of, capturing the three-dimensional image of the user's posture, the 3-D scanner may also capture a three-dimensional image of equipment on the user's workstation. By way of example, and without limitation, the 3-D scanner may capture a three-dimensional image of a telephone or a lamp on the user's desk. Analysis of the locations of these devices may reveal that the user is stretching to reach these devices. It may be suggested that the user re-position these items into a location that results in better ergonomic outcome for the user. An algorithm is provided in step 404, which is configured to operate on the user's computer. The algorithm is adapted to receive the three-dimensional image of the user's posture and/or the three dimensional image of the user's workspace and equipment situated thereon.

As shown in step 405, the algorithm is also adapted to receive other information relating to the user's ergonomic characteristics. The other information relating to the user's ergonomic characteristics may be input by the user. By way of example, and without limitation, the other information relating to the user's ergonomic characteristics may include information about ergonomic products installed at the user's desk, information about the user's posture and position relative to the workspace, information about the user's health history or information about pain experienced by the user.

In step 406, the algorithm analyzes the three dimensional user's posture and/or the three dimensional image of the user's workspace and equipment situated thereon and the other information relating to the user's ergonomic characteristics. In this step, the algorithm may compare the three dimensional image of the user's posture to a model of ergonomically correct posture and note deficiencies in the user's posture as compared to the model posture. Similarly, the algorithm may identify the user's position with respect to equipment situated on the user's desk, and note where the positioning of the equipment causes the user's posture to deviate from the model posture. Having noted the deficiencies in the user's posture, based on the three dimensional data, the algorithm may further refine the analysis of the user's ergonomic characteristics by factoring data supplied by the user. For example, if the user indicates that his or her health history makes the user susceptible to ergonomic injury, the algorithm would note that the user is at a heightened risk. If the user indicated that he or she was currently experiencing pain, the algorithm would note that the user is likely currently experiencing ergonomic injury, and had a very high risk for same.

In step 407, the algorithm provides a report on the user's ergonomic attributes. The user's ergonomic attributes may include a description of the user's ergonomic deficiencies, a description of suggested equipment to remedy the user's ergonomic deficiencies and a description of suggested behavioral changes to remedy the user's ergonomic deficiencies. One of ordinary skill in the art will appreciate that the report can contain any relevant information about the user's ergonomic situation that will assist in identifying those users with ergonomic problems and addressing ways to correct those ergonomic problems. Also, the report may rank multiple users by the amount of risk of ergonomic injury each user has. Thus, users with high risk of ergonomic injury can be identified and an ergonomist other appropriate person in the organization can intervene to address the user's ergonomic deficiencies. The report on the use ergonomic attributes may be provided to the user, the user's supervisor, and/or a person designated to oversee ergonomic issues in the user's organization.

It will be appreciated by those of ordinary skill in the art that, while the forgoing disclosure has been set forth in connection with particular embodiments and examples, the disclosure is not intended to be necessarily so limited, and that numerous other embodiments, examples, uses, modifications and departures from the embodiments, examples and uses described herein are intended to be encompassed by the claims attached hereto. Various features of the disclosure are set forth in the following claims.

I claim:

1. A system comprising:
a 3-D scanner adapted for connection to a user's workspace computer, said 3-D scanner comprising:
an infrared light source;
an infrared light detector;
a three-dimensional camera;
at least one RGB camera;
and wherein said 3-D scanner is operatively connected to a user's computer, and said 3-D scanner captures a three-dimensional image of a user's posture;
an algorithm configured to operate on the user's computer, said algorithm adapted to receive the three-dimensional image of the user's posture and to receive other information relating to the user's ergonomic characteristics,
said algorithm being further configured to analyze the user's posture and the other information relating to the user's ergonomic characteristics and to provide a report on the user's ergonomic attributes.

2. The system of claim 1, wherein the 3-D scanner captures the three-dimensional image of the user's posture by using a technique selected from the group consisting of the time-of-flight technique, the structured light technique and the stereo image technique.

3. The system of claim 1, wherein the 3-D scanner includes two or more cameras that view a scene at different angles to provide depth information.

4. The system of claim 1, wherein the other information relating to the user's ergonomic characteristics comprises input from the user about ergonomic products installed at the user's desk.

5. The system of claim 1, wherein the other information relating to the user's ergonomic characteristics comprises input from the user about the user's posture and position elative to the workspace.

6. The system of claim 1, wherein the other information relating to the user's ergonomic characteristics comprises input from the user about the user's health history.

7. The system of claim 1, wherein the other information relating to the user's ergonomic characteristics comprises input from the user about pain experienced by the user.

8. The system of claim 1, wherein the algorithm is stored remotely from the user's computer and accessed by the user's computer from the remote location.

9. The system of claim 1, wherein the report on the user's ergonomic attributes comprises at least one of a description of the user's ergonomic deficiencies, a description of suggested equipment to remedy the user's ergonomic deficiencies and a description of suggested behavioral changes to remedy the user's ergonomic deficiencies.

10. The system of claim 1, wherein the report on the user's ergonomic attributes is provided to at least one of the user, a user's supervisor, and a person designated to oversee ergonomic issues in the user's organization.

11. A method comprising:
providing a 3-D scanner, said 3-D scanner comprising: an infrared light source; an infrared light detector; a three-dimensional camera; at least one RGB camera;
operatively connecting the 3-D scanner to a user's computer;
capturing by the 3-D scanner a three-dimensional image of a user's posture;
providing an algorithm configured to operate on the user's computer, said algorithm adapted to receive the three-dimensional image of the user's posture and to receive other information relating to the user's ergonomic characteristics,
analyzing by the algorithm the user's posture and the other information relating to the user's ergonomic characteristics and
providing a report on the user's ergonomic attributes.

12. The method of claim 11 wherein the 3-D scanner captures the three-dimensional image of the user's posture by using a technique selected from the group consisting of the time-of-flight technique, the structured light technique and the stereo image technique.

13. The method of claim 11, wherein the 3-D scanner includes two or more cameras that view a scene at different angles to provide depth information.

14. The method of claim 11, wherein the other information relating to the user's ergonomic characteristics comprises input from the user about ergonomic products installed at the user's desk.

15. The method of claim 11, wherein the other information relating to the user's ergonomic characteristics comprises input from the user about the user's posture and position relative to the workspace.

16. The method of claim 11, wherein the other information relating to the user's ergonomic characteristics comprises input from the user about the user's health history.

17. The method of claim 11, wherein the other information relating to the user's ergonomic characteristics comprises input from the user about pain experienced by the user.

18. The method of claim 11, wherein the algorithm is stored remotely from the user's computer and accessed by the user's computer from the remote location.

19. The method of claim 11, wherein the report on the user's ergonomic attributes comprises at least one of a description of the user's ergonomic deficiencies, a description of suggested equipment to remedy the user's ergonomic deficiencies and a description of suggested behavioral changes to remedy the user's ergonomic deficiencies.

20. The method of claim 11, wherein the report on the user's ergonomic attributes is provided to at least one of the user, a user's supervisor, and a person designated to oversee ergonomic issues in the user's organization.

21. A system comprising:
a 3-D scanner adapted for connection to a user's workspace computer, said 3-D scanner comprising:
an infrared light source;
an infrared light detector;
a three-dimensional camera;
at least one RGB camera;
and wherein said 3-D scanner is operatively connected to a user's computer, and said 3-D scanner captures a three-dimensional image of a user's workstation and equipment situated thereon;
an algorithm configured to operate on the user's computer, said algorithm adapted to receive the three-dimensional image of the user's workstation and equipment situated thereon and to receive other information relating to the user's ergonomic characteristics,
said algorithm being further configured to analyze the user's workstation and equipment situated thereon and the other information relating to the user's ergonomic characteristics and to provide a report on the user's ergonomic attributes.
